(19) United States
(12) Patent Application Publication
(10) Pub. No.: US 2006/0015163 A1
(43) Pub. Date: Jan. 19, 2006

(54) Lead extender for implantable device

(75) Inventor: Ward M. Brown, La Crosse, WI (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: Team Brown Enterprises, LLC, La Crosse, WI (US)

(21) Appl. No.: 11/183,428

(22) Filed: Jul. 18, 2005

Related U.S. Application Data
(60) Provisional application No. 60/589,148, filed on Jul. 19, 2004.

(51) Int. Cl. A61N 1/05

(52) U.S. Cl. 607/116

(57) ABSTRACT

A device for extending the length of the existing leads of an implantable device. The lead extenders are constructed and arranged for placement on the existing leads without removal thereof, significantly simplifying installation and obviating the need for contact with the organ connected to the distal ends of the leads. The lead extenders are provided at varying length to provide a physician with the most appropriate device for the particular application.

### CROSS REFERENCE TO RELATED APPLICATIONS

This application claims the benefit of prior U.S. Application No. 60/589,148, which was filed on Jul. 19, 2004.

### BACKGROUND OF THE INVENTION

The present invention generally relates to implantable electro-stimulation devices for use in the human body. More specifically, the present invention relates to the leads that extend from the device to the organ being stimulated.

The human heart normally maintains its own intrinsic rhythm in order to consistently pump a proper supply of blood throughout the body's circulatory system. However, some people are afflicted with irregular cardiac rhythms, or cardiac arrhythmias, resulting in diminished blood circulation. Drug therapy is one mode of treatment for cardiac arrhythmias. Unfortunately, drug therapy is not effective for treating all cardiac arrhythmias. Hence, alternative modes of treatment including implantable electrotherapy devices, such as pacemakers and defibrillators, are utilized.

Patients with bradyarrythmias, or symptomatic or slow beating of the heart, are often treated with pacemakers. These devices deliver timed sequences of low energy electrical stimuli to the heart via leads having one or more electrodes placed about the heart. With proper timing of the electrical stimuli, heart contractions are regulated such that the heart contracts at a proper rate, greatly improving blood supply throughout the body's circulatory system.

Patients with malignant tachyarrhythmia, or potentially life threatening fast beating of the heart, are often treated with implantable cardioverter defibrillators. These devices deliver high-energy electrical stimuli called defibrillation countershock to the heart. The countershock interrupts the tachyarrhythmia allowing the heart to establish a perfusing rhythm, which allows the heart to completely fill with blood before pumping. Other implantable electrotherapy devices include pacer/defibrillators, which combine the functions of pacemakers and defibrillators, drug delivery devices, and other systems designed for diagnosing and treating arrhythmias.

In addition to the above heart conditions that are treated with electrical stimulation signals, various muscle and nerve conditions also benefit from electrical stimulation. For example, electrical signals may be used for pain management, where signals effect nerve system reaction. Further, electrical systems also include muscular stimulation devices, which provide appropriate signals to the body to aid in injury recovery. In another example, drug delivery is achieved using electrical signals to “drive” certain drugs into the body.

Conventional implantable electrode leads used together with implantable electrotherapy devices are commonly known. An implantable electrode lead is generally comprised of at least one electrode for supplying an electrical stimulation pulse or sensing an electrically evoked response of the heart, an electrical connector for connecting the electrode lead to an implantable electrotherapy device, and a lead body inserted between the electrode and the electrical connector for transmitting an electrical signal between the electrode and the implantable electrotherapy device.

Implantation surgery involves a first step of connecting distal ends of the leads to the organ to be treated. In the case of the heart, an organ that violently and continually beating, the attachment between the lead and the muscle must be able to endure both the physical movement of the heart and the changing density of the heart muscle as it flexes and relaxes.

The next step is to find a suitable location to implant the device. Defibrillators are usually placed in the chest wall under the collarbone. This location provides a protected place where the device won't interfere with heart and lung movement.

The proximal ends of the leads are then secured to the device and the entrance wound is closed. The lengths of the leads vary based on the size of the patient. It is undesirable to have leads that are somewhat taught because the chances of them becoming dislodged are increased. However, leads that are too long are also undesirable because due to lead losses inherent in all electrical leads. Furthermore, excessive length can result in migration of the leads to areas where they become affected by moving organs, etc. If the attending physician determines that the leads are too short after attaching the distal ends thereof to the organ, the physician must presently remove the leads and replace them with longer leads, thereby extending the procedure and significantly increasing the risks associated therewith.

Though the occurrence of a situation where it is determined that longer leads are required can be minimized through careful planning, reality dictates that such instances are never eliminated. Problems may arise despite careful planning that require the relocation of an implantable device, either upon initial placement of during a follow-up procedure. There is thus a need for a device that allows the lengthening of leads after the distal ends thereof have been attached to a recipient organ.

### BRIEF SUMMARY OF THE INVENTION

The present invention is directed to fulfilling the identified need by providing a lead system whereby lead extension may be attached to a proximal end of the lead of an insertable device that has already been attached to a recipient organ. The present invention includes an attachment mechanism and lead extensions of various lengths such that an optimal lead length can be achieved even after leads have been attached to the target organ. The lead extensions are useful during initial implantation of the device and during subsequent device relocation procedures.

The present invention includes a lead extension which has connectors on its ends which are configured to mate with the existing lead and the implantable device. In this way, the attending physician can simply attach the extension to the lead, thus extending the overall distance between an installed electrode and the implantable device. As clearly anticipated, this is much more desirable than removing the attached electrode so that a longer lead can be used.

The connectors on the extension also include sufficient features to provide a secure attachment. This may include a snap fit structure, or may include set screws, to ensure that the connection is solidly made. Further, an insulating covering is provided which can cover the connecting elements and providing further protection.

### BRIEF DESCRIPTION OF THE DRAWINGS

FIG. 1 is a perspective view of an embodiment of the system of the present invention; and,

FIG. 2 is a perspective view of an embodiment of a lead extension of the present invention.

### DETAILED DESCRIPTION OF THE INVENTION

Referring now to the Figures, there is a shown a system 10 of the present invention. The system includes an implantable device 12, a lead 14 for attachment to a recipient organ, and a lead extension 16 for placement between the device 12 and the lead 14, thereby effectively increasing the length of the lead 14

The implantable device 12 may be any implantable device using leads. The most commonly recognized implantable devices include pacemakers and defibrillators. The device includes at least one male or female connector 18, to which a lead 14 or extension 16 may be attached. For purposes of discussion, an implantable cardioverter defibrillator will be used as an example of an implantable device 12.

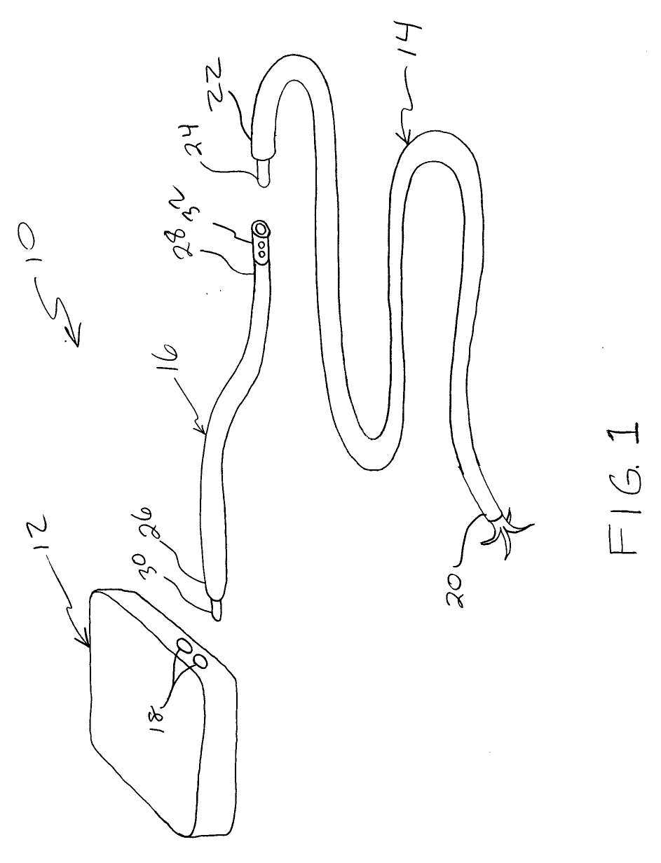

The leads 14 include a distal end 20, for attachment to the recipient organ, in this example the heart (not shown). Opposite the distal end 20 is a proximal end 22. The proximal end 22 includes a female or male connector 24, attachable to the male or female (respectively) connector 18 of the device 12. The example shown in FIG. 1 includes a device 12 having standard female connectors 18, into which leads 14 having standard male connectors 24 at their proximal ends 22 may be plugged.

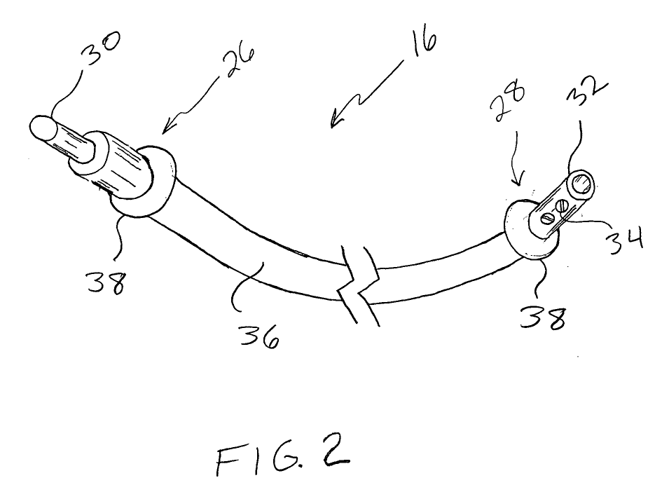

The lead extensions 16 of the present invention are constructed and arranged such that a lead 14 may be unplugged from a device 12 and a lead extension 16 may be placed between the device 12 and the lead 14. Thus, the lead extension 16 includes a proximal end 26 and a distal end 28. The proximal end 26 includes a connector 30 capable of being connected with a connector 18 of the device 12. In the example of FIG. 1, and shown in greater detail in FIG. 2, the connector 30 at the proximal end 26 of the lead extension 16 is a male connector that is physically similar, if not identical, to the connector 24 of the lead 14. The distal end 28 includes a connector 32 that is configured to mate with the connector 24 at the proximal end 22 of the lead 14. Preferably, the connector 32 includes a securing device 34, such as setscrews, a snap connector, or the like, to prevent unintentional disconnection from the lead 14.

The lead extension 16 may be of any length but it is envisioned that extensions are provided in various incremental lengths such as 5 cm, 10 cm, 15 cm, etc. As another feature of the present invention extension 16 is covered in a sleeve 36 having rolled ends 38 that can be unrolled after installation to cover the connectors. The sleeve 36 may be constructed of the same material as that used to cover the leads 14. This rolled end structure 38 provides the ability to insulate and protect the connection between leads 14 and extension 16 when implemented. This obviously provides protection to the connection and insures that undesired signals are not transmitted to other parts of the body.

In order to provide more convenience to the physician, the extensions may be provided as a kit. More specifically, the kit may include a number of extensions of varying length. Thus, during procedures, the physician would have the opportunity to simply select the extension that best fits the particular needs of the patent.

It is contemplated that features disclosed in this application can be mixed and matched to suit particular circumstances. Various other modifications and changes will be apparent to those of ordinary skill in the art without departing from the spirit and scope of the present invention. Accordingly, reference should be made to the claims to determine the scope of the present invention.

What is claimed is:

1. A lead extender for use with an implantable device system having a main lead of a predetermined length designed to be connected between a system electrode and an implantable device, the main lead having a device/lead connector on a proximal end to accommodate the connection to the implantable device, the lead extender comprising:
a lead extension made up of a connecting wire with a distal end and a proximal end, the lead extension having an insulating covering over the connecting wire;
a first connector attached to the distal end of the lead extension, the first connector configured to mate with the device/lead connector so as to allow easy connection between the main lead and the lead extension, the first connector also having a configuration capable of providing a secure connection; and
a second connector attached to the proximal end of the lead extension, the second connector being substantially identical to the device/lead connector to provide connection between the lead extension and the implantable device.

2. The lead extender of claim 1 wherein the first connector includes a set screw to provide a secure connection between the extension and the main lead.

3. The lead extender of claim 1 wherein the first connector includes a snap-fit structure to provide the secure connection between the first connector and the main lead.

4. The lead extender of claim 1 wherein the protective covering further extends a sufficient distance to cover the first connector when attached to the main lead.

5. The lead extender of claim 1 wherein the lead extension is of a predetermined length.

6. The lead extender of claim 1 wherein the first connector is a female type connector and the second connector is a male type connector.

7. A lead extender kit for use with an implantable device system, the implantable device system including a main lead for connection between an electrode and an implantable device, the main lead having a device/lead connector on a proximal end to accommodate the connection to the implantable device, the lead extender kit comprising:
a plurality of lead extensions, each lead extension having a varying length so as to be selectable to provide a desired extension length to the implantable device system, each lead extension further made up of a connecting wire with a distal end and a proximal end, each lead extension further having a first connector attached to the distal end of the lead extension, the first connector configured to mate with the device/lead connector so as to allow easy connection between the main lead and the main extension, the first connector also having a configuration capable of providing a secure connection and a second connector attached to the proximal end of the lead extension, the second connector being substantially identical to the device/lead connector to provide connection between the lead extension and the implantable device.

8. The lead extender kit of claim 7 wherein the varying lengths of the plurality lead extensions vary by a predetermined interval.

9. The lead extender kit of claim 8 wherein the predetermined interval is 5 centimeters.

10. The lead extender kit of claim 7 wherein the first connector of each lead extension includes a set screw to provide a secure connection between the extension and the main lead.

11. The lead extender kit of claim 7 wherein the first connector of each lead extension includes a snap-fit structure to provide the secure connection between the first connector and the main lead.

12. The lead extender kit of claim 7 wherein each lead extension further includes a protective covering to insulate the connecting wire, wherein the protective covering further extends a sufficient distance to cover the first connector.

13. The lead extender kit of claim 7 wherein the first connector of each lead extension is a female type connector and the second connector is a male type connector.

14. A method for easily extending the connection distance between an implantable device and an associated electrode, the method comprising;
selecting a lead extension of an appropriate length;
attaching the lead extension to an existing lead which is configured for attachment to the implantable device via a lead/device connection, wherein the lead extension made up of a connecting wire with a distal end and a proximal end and includes a first connector attached to the distal end of the lead extension, the first connector configured to mate with a lead/device connector on the existing lead so as to allow easy connection between the existing lead and the lead extension; and
attaching the lead extension to the implantable device using a second connector attached to the proximal end of the lead extension, the second connector being substantially identical to the device/lead connector to provide connection between the lead extension and the implantable device.

15. The method of claim 14 wherein the first connector further includes a set-screw device for providing the secure attachment, and wherein the method further comprised securing the connection via the set-screw device.

16. The method of claim 14 further comprising extending a protective covering which is provided on the lead extension so as to also cover the connection between the first connector and the existing lead.

17. A lead extender for use with an implantable device system, the implantable device system having a main lead of a predetermined length designed to be connected between a system electrode and an implantable device, the main lead having a device/lead connector on a proximal end to accommodate the connection to the implantable device, the lead extender comprising:
a lead extension made up of a connecting wire with a distal end and a proximal end, the lead extension having an insulating covering over the connecting wire;
a first connector attached to the distal end of the lead extension, the first connector configured to mate with the device/lead connector so as to allow easy connection between the main lead and the lead extension, wherein the insulating covering of the first connector has a length sufficient to also cover the first connector when attached to the main lead thus providing insulation to the connection; and
a second connector attached to the proximal end of the lead extension, the second connector being substantially identical to the device/lead connector to provide connection between the lead extension and the implantable device.

18. The lead extender of claim 17 wherein the first connector includes a set screw to provide a secure connection between the extension and the main lead.

19. The lead extender of claim 17 wherein the first connector includes a snap-fit structure to provide the secure connection between the first connector and the main lead.
